(19) United States
(12) Patent Application Publication
(10) Pub. No.: US 2021/0128138 A1
(43) Pub. Date: May 6, 2021

(54) ADJUSTABLE AND SELF-LOCKING SUSPENSORY FIXATION DEVICES

(71) Applicant: Mason James Bettenga, Memphis, TN (US)

(72) Inventor: Mason James Bettenga, Memphis, TN (US)

(21) Appl. No.: 17/090,644

(22) Filed: Nov. 5, 2020

Related U.S. Application Data
(60) Provisional application No. 62/931,356, filed on Nov. 6, 2019.

(51) Int. Cl. A61B 17/04

(52) U.S. Cl. CPC A61B 17/0401; A61B 2017/0404; A61B 2017/0464; A61B 2017/0446; A61B 2017/0414

(57) ABSTRACT

A suspensory fixation device includes a standing block at one end thereof and a set of at least two adjustable loops at the second end thereof. The standing block includes a rigid fixation member having a plurality of apertures forming at least one sheave and a becket. A single reeved fall, rigid fixation member, adjustable loops, and sliding knot form a block and tackle system. A suspensory fixation button includes a rigid body having a top side and a bottom side, a plurality of lobes extending radially from the center of the body, and at least one aperture through the body near the root of a lobe. A method of coupling a suspensory fixation button to a set of adjustable flexible loops includes passing each adjustable loop over an edge of an extension of the button from the top side to the bottom side.

### CROSS REFERENCE TO RELATED APPLICATIONS

This application claims priority to U.S. Provisional App. No. 62/931,356 filed Nov. 6, 2019, titled ADJUSTABLE AND SELF-LOCKING SUSPENSORY FIXATION DEVICES, herein incorporated by reference in its entirety for all purposes.

### FIELD OF THE INVENTION

The present invention relates to devices and methods for surgical procedures requiring adjustable levels of tension between anatomical structures.

### BACKGROUND

Fixation devices using flexible cables in combination with rigid fixation bodies intended to anchor tissue under tension or provide compression across surfaces have become increasingly utilized to repair anatomical structures. It is preferred that such devices provide the user with a feature to enable the adjustment of the level of tension between anatomical structures constrained by such devices, but do not require the user to form a sliding, adjustable knot in the surgical theater. Fixation devices, sometimes referred to as knotless, have been developed which incorporate a pulley system with a self-locking section having the lines of flexible cables woven through cavities in its interior, utilizing the friction of the tortuous path as the one-way adjustment mechanism. However, systems using this method of construction are limited in adjustable working length by the proportion of the locking section to the total length of the adjustable section.

A second limitation of such devices currently available is the lack of secondary fixation means for securing a tissue graft directly to the adjustable system, necessary for a variety of reasons. The user may prefer additional construct strength or eliminate the motion of tissue with respect to the suspensory device. Current methods require the user to purchase additional implants which increase cost and add additional surgical time. For example, procedures replacing damaged ligaments comprised of two bundles of tissue, each adjusted for a different level of tension at various levels of joint flexion, such as anterior cruciate or posterior cruciate ligaments of the knee, may be constructed with allograft or autograft sourced from tendinous sources such as hamstring, semitendinosus, or gracilis donor sites, and fashioned into two separate grafts. Each graft is formed with suspensory fixation devices and tensioned in separate bone tunnels. Though this double-bundle repair technique is considered the best anatomical solution, the surgeon must weigh the cost of the surgical time to harvest the graft tissues and prepare the bone tunnels along with the additional implant consumables compared to single-bundle techniques, since a consolidated implant solution for creating a double-bundle repair using a single graft is not presently available. Choosing a non-anatomical reconstruction may have adverse effects to the patient regarding the life or performance of the repair. Thus, the need for a solution to reduce cost and surgical time to complete an anatomical, double-bundle repair with a single graft is clearly needed.

Suspensory fixation devices are also employed to provide tension across multiple bony structures to stabilize fibrous joints. Syndesmosis joints, such as the tibiofibular joint of the ankle, or a diarthrodial joints, such as the acromioclavicular (AC) joint in the shoulder, are often repaired by passing adjustable loops of a suspension system from one side of a single aperture formed through the bones to exit the opposing side. Two common methods are used in these techniques. Firstly, a pre-assembled suspension system, where two buttons are provided to the user affixed to an adjustable loop, or secondly, a system which requires the user to assemble a second button onto the adjustable implant intraoperatively may be used. One problem encountered with a system that is pre-assembled, is that the tunnel diameter must be sufficient for one of the rigid devices to pass through. As the rigid device size increases due to larger loads, tunnel diameter increases proportionally. The increase in the amount of bone removed is of particular concern for the practitioner, as this may lead to a weakening of the bony structure potentially leading to an overload failure. One concern with the solution of attaching a button to the loops after being passed through the bone tunnel is the length of surgical time consumed to securely affix the rigid member onto the loops. Once affixed, great care must be taken to prevent the fixation button from disassociating from the loops when passed through tissue retrograde toward the bone aperture. Often, the surgeon will adjust and tension the construct under direct visualization further adding cost and time to the procedure. Therefore, a suspensory fixation system which solves the aforementioned problems is clearly needed.

### BRIEF SUMMARY OF THE INVENTION

In one embodiment of the invention a suspensory fixation device is provided, comprising a block at a first end thereof, the block comprising a rigid fixation member having a first end and a second end and a plurality of apertures therebetween forming at least one sheave and at least one becket, a tackle comprising a single fall reeved through the apertures of the standing block to form a set of at least two adjustable loops at the second end thereof, the fall comprising a flexible cable with a first end and a second end, wherein at least one portion of the fall engages at least one sheave of said standing block, a sliding knot, formed in the fall and adjustable in one direction, the sliding knot having at least one portion of the fall forming a locking loop on one side of the rigid fixation member and engaging with a becket, both ends of the fall passing through the locking loop, and a haul operable to adjust the sliding knot, wherein the adjustment of the sliding knot reduces the diameter of the set of loops, and the portion of the reeved fall engaging a becket on the rigid fixation member forms the standing part of the block and tackle system. Also in one embodiment, any sheave in the rigid fixation member may function as a becket. Also in one embodiment, the knot further comprises a transport limb, wherein a force applied to the transport limb will not cause the set of loops to change diameter. Also in one embodiment, the suspensory fixation device further comprises a second rigid fixation member at the second end thereof, wherein the fall is reeved through apertures and over at least one sheave of the second fixation member, and the second rigid fixation member forms a running block in the block and tackle system. Also in one embodiment, the rigid fixation member is curved along its long axis, wherein the radius of curvature allows the rigid fixation member to be congruent with an anatomic structure when approximated to said anatomic structure. Also in one embodiment, the suspensory fixation device further comprises a needle coupled to at least one adjustable loop of the set and may be moveable on at least one adjustable loop. The suspensory fixation device may further comprise a grasping element formed on at least one end of the fall.

In another aspect of the invention, a suspensory fixation device is provided, comprising a rigid fixation member, a flexible cable fashioned into at least one adjustable loop, the rigid fixation member coupled to at least one adjustable loop at the first end; and a flexible cable fashioned into at least one adjustable loop, wherein the rigid fixation member coupled to at least one adjustable loop at the first end, and a graft support member comprising a flexible cable integrated onto at least one adjustable loop at the second end, the flexible cable having a first end and a second end, and a hollow portion therebetween, wherein a portion of at least one adjustable loop is contained within said hollow portion of the graft support member. Also in one embodiment, the graft support member further comprises a needle affixed to at least one end of the flexible cable. Also in one embodiment, the graft support member further comprises a closed loop formed by joining the ends of the graft support member and a needle coupled to said closed loop, wherein the needle may be movable on the closed loop.

In another example, a method of forming an adjustable, self-locking knot is provided, comprising the steps: (a) reeving a single fall through the apertures of at least one rigid fixation member to form a tackle, the rigid fixation member comprising a first end and a second end and a plurality of apertures therebetween through a first side to a second side forming at least one sheave and at least one becket, the fall comprising a flexible member with a first end and a second end, (b) forming a locking loop with a portion of the fall on one side of the rigid fixation member through which the ends of the fall are passed, (c) engaging a portion of the fall with a becket on the rigid fixation member, wherein the engaged portion functions as the standing part in a block and tackle system, and (d) passing both ends of the fall through the locking loop, wherein the end of the fall forming the locking loop also engages a becket, and the locking loop is formed prior to the end engaging the becket.

In another embodiment, a method of coupling a suspensory fixation button to a set of adjustable flexible loops is provided, comprising the steps: (a) passing a set of adjustable loops through an aperture on a suspensory fixation button from a first side to a second side, the suspensory fixation button comprising a rigid body having a first side and a second side and at least a first lobe and a second lobe, the lobes extending radially from the body and at least one aperture formed through the body between the first lobe and the second lobe from the first side to the second side, the set of adjustable loops comprising a first adjustable loop and a second adjustable loop, (b) passing the first adjustable loop over an edge of the first lobe to arrive on the first side of the first lobe, and (c) passing the second adjustable loop over an edge of the second lobe to arrive on the first side of the second lobe. Also in one embodiment, the method further comprises passing the set of adjustable loops through tissue. Also in one embodiment, the suspensory fixation button further comprises a sleeve extending from the first side of the rigid body concentric with the aperture. Also in one embodiment, the suspensory fixation button comprises at least one lobe having a radius of curvature, wherein said lobe forms a concave geometry on the first side of the device. The suspensory fixation button may also provide a retrieval means comprising a flexible member connected to the suspensory fixation button.

### BRIEF DESCRIPTION OF THE DRAWINGS

The foregoing and other objects, features, and advantages will be apparent from the following more elaborate description of the embodiments, as illustrated in the accompanying drawings in which like reference characters refer to the same parts throughout the different views. The drawings are not necessarily to scale, emphasis instead being placed upon illustrating the principles of the embodiments. For a detailed description of example embodiments, reference will now be made to the accompanying drawings in which:

FIG. 1 is a perspective view of an adjustable, self-locking suspensory fixation device, according to an embodiment of the present invention;

FIG. 2A shows a perspective view of the rigid fixation body of FIG. 1, in accordance with the disclosure;

FIG. 2B illustrate top and side views of the rigid fixation body of FIG. 1, in accordance with the disclosure;

FIGS. 3A-3D show perspective and top views, respectively, of alternative embodiments of the rigid fixation member of FIG. 1, in accordance with the disclosure;

FIG. 4 illustrates exemplary steps for reeving a fall through the rigid fixation member of FIG. 1 to form adjustable loops extending from one side of the rigid fixation member, according to an embodiment of the present invention;

FIG. 5 illustrates exemplary steps for forming a sliding and self-locking knot in the reeved fall of FIG. 4, according to an embodiment of the present invention;

FIG. 6 illustrates alternative exemplary steps for forming the knot of FIG. 5, in accordance with the disclosure;

FIG. 7A illustrates an optional step in forming the knot of FIGS. 5-6 to enable orientation of the rigid member of FIG. 1 along its long axis for transportation through tissue, in accordance with at least one embodiment disclosed;

FIG. 7B is a perspective view of the suspensory fixation device of FIG. 1 integrated with a ligament and oriented for transportation through a bone tunnel, according to an embodiment of the present invention;

FIGS. 8A-8D illustrate alternative embodiments of the construct of FIG. 4, in accordance with the disclosure;

FIG. 9A is a perspective view of a rigid fixation device, according to an embodiment of the present invention;

FIG. 9B is a side view of the rigid fixation device of FIG. 9A, in accordance with the disclosure;

FIG. 9C is a second perspective view of the rigid fixation device of FIG. 9A, in accordance with the disclosure;

FIG. 9D is a section view of the rigid fixation device of FIG. 9A, in accordance with the disclosure;

FIG. 10 illustrates the exemplary steps for reeving adjustable loops onto the rigid fixation member of FIG. 9A, according to an embodiment of the present invention;

FIG. 11 illustrates alternative exemplary steps for reeving adjustable loops onto the rigid fixation member of FIG. 9A, according to an embodiment of the present invention;

FIG. 12 illustrates exemplary steps for forming a sliding and self-locking knot in the reeved fall of FIG. 10, in accordance with at least one embodiment disclosed;

FIG. 13 is a perspective view of the adjustable, self-locking fixation device of FIG. 1 integrated with a flexible graft support member, according to another embodiment of the present invention;

FIG. 14 illustrates the components of FIG. 13 oriented for assembly, in accordance with the disclosure;

FIG. 15 illustrates exemplary steps for assembling the embodiment of FIG. 13, according to an embodiment of the present invention;

FIGS. 16A-16C illustrate alternative embodiments of the construct of FIG. 4, integrated with a second rigid fixation member, in accordance with the disclosure;

FIG. 17 illustrates an alternative embodiment of the construct of FIG. 1, integrated with a needle, according to an embodiment of the present invention;

FIG. 18A illustrates the assembly of FIG. 13 further provided with graft tissue (ligament for example) placed over the flexible graft support member, in accordance with the disclosure;

FIG. 18B illustrates the assembly of FIG. 18A with the graft member secured to the graft tissue, according to an embodiment of the present invention;

FIG. 19 is a perspective view of the flexible, adjustable, self-locking fixation device of FIG. 13 with surgical needles affixed to the ends of the graft support member, according to an embodiment of the present invention;

FIGS. 20A-20C illustrate exemplary steps forecuring the graft support member of FIG. 19. to tissue (ligament for example), in accordance with at least one embodiment disclosed;

FIG. 21 is a perspective view of the suspensory fixation device of FIG. 13 integrated floating surgical needle on a loop formed in the graft support member, according to an embodiment of the present invention;

FIGS. 22A-22B illustrate exemplary steps for assembling the needle onto the loop of the graft support member of FIG. 21, in accordance with at least one embodiment disclosed;

FIGS. 23A-23B illustrate exemplary steps for securing the suspensory fixation device of FIG. 21. to tissue (ligament for example), according to an embodiment of the present invention;

FIG. 24 illustrates the device of FIG. 21 stitched to tissue (ligament for example), in accordance with at least one embodiment disclosed;

FIG. 25A shows the components for a user-assembled suspension fixation system, in accordance with at least one embodiment disclosed;

FIG. 25B illustrates the assembled form of the devices of FIG. 25A, in accordance with at least one embodiment disclosed;

FIG. 26 show top, front, side, and perspective views of a rigid fixation member of FIG. 25A, in accordance with at least one embodiment disclosed;

FIG. 27 illustrates exemplary stems for assembling the suspensory fixation system of FIGS. 25A-25B, in accordance with at least one embodiment disclosed;

FIGS. 28A-28B are close-up, perspective views of the rigid fixation device of FIG. 26 assembled onto the flexible, adjustable loops the suspensory fixation system of FIGS. 25A-25B, in accordance with at least one embodiment disclosed;

FIGS. 29A-29D illustrate exemplary steps for using the devices of FIG. 25 to reduce an acromioclavicular injury, according to an embodiment of the present invention;

FIGS. 30A-30C illustrate the orientation of a series of security knots tied after final adjustment of the suspensory fixation system of FIGS. 25A-25B upon completion of the steps illustrated in FIGS. 29A-29D, according to an embodiment of the present invention.

While the invention is amenable to various modifications, permutations, and alternative forms, specific embodiments have been shown by way of example in the drawings and are described in detail below. The intention, however, is not to limit the invention to the embodiments described. The invention is intended to cover all modifications, equivalents, and alternatives falling within the scope of the invention as defined by the appended claims.

### DETAILED DESCRIPTION OF THE INVENTION

The inventor provides a unique device for an adjustable suspensory fixation implant. The present invention is described in enabling detail in the following examples, which may represent more than one embodiment of the present invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

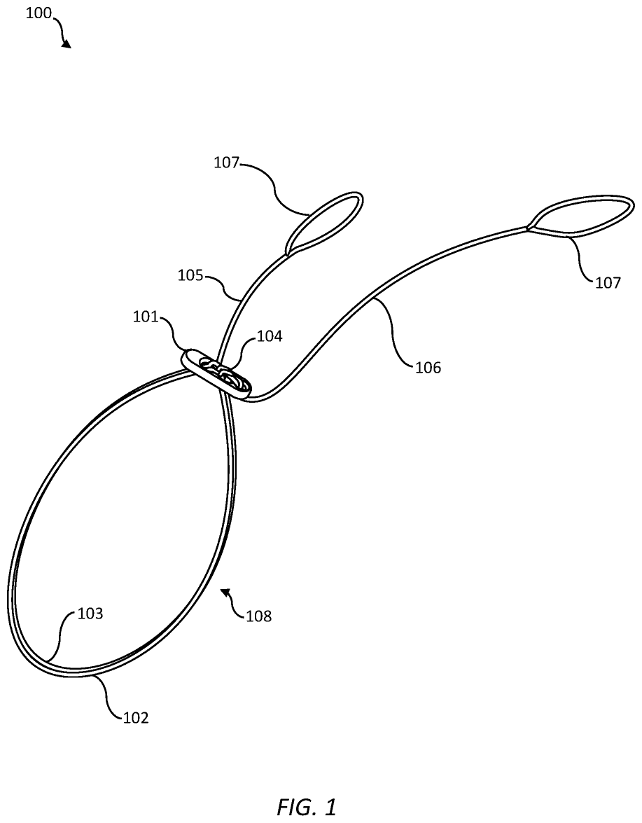

Referring now to FIG. 1, one embodiment of a suspensory fixation device 100 is shown. In this example, suspensory fixation device 100 is comprised of a rigid member 101 and a single flexible fall 108 reeved onto rigid member 101 such that a first adjustable loop 102 and a second adjustable loop 103, having a length generally similar to adjustable loop 102, extend through selected apertures formed in rigid member 101. may be manufactured from a monofilament or a plurality of biologically compatible fibers braided into either a solid or hollow cable with suitable strength tailored for a particular repair indication. Non-limiting examples of permanent materials for are ultra-high molecular weight polyethylene (UHMWPE), polyester, polypropylene, carbon fiber, metal wires, or silk. Non-limiting examples of resorbable materials which may be used to manufacture having limited lifespan in the body are polylactic acid (PLA), polyglycolic acid (PGA), and polygalactin. Rigid member 101 may be manufactured from biologically compatible solid materials with suitable strength for a particular indication. Non-limiting examples of such materials are titanium, alloys of titanium, alloys of stainless steel, polyether ether ketone (PEEK), or other fiber reinforced thermoset or thermoplastic composites. Rigid member 101 may also be manufactured from resorbable biomaterials if the user desires resorption of the implant.

Suspensory fixation device 100 incorporates a sliding self-locking knot 104 such that when a haul 105 is manipulated by the user, adjustable loops 102 and 103 will reduce in diameter and cause objects connected to adjustable loops 102 and 103 to be brought into closer proximity to rigid member 101, provided the loops are free to slide through or around said connected objects. A unique method of forming knot 104 is disclosed in one embodiment of the present invention such that when the user stops applying a pulling force to haul 105 during the reduction process, adjustable loops 102 and 103 will remain at the adjusted state and not return to a larger diameter. In this embodiment, reeved fall 108, rigid member 101, and knot 104 form a reverse luff tackle rove to advantage with the set of adjustable loops 102 and 103 functioning as the moveable pulley and rigid member 101 functioning as the standing block of said tackle.

Furthermore, FIG. 1 also illustrates a transport limb 106 which allows the user to manipulate suspensory fixation device 100, and objects connected to suspensory fixation device 100, with a pulling force without changing the diameter of adjustable loops 102 and 103. Transport limb 106 can be woven through rigid member 101 in various advantageous ways to cause rigid member 101 to orient in preferred directions during transport, such as a long or short axis parallel to the direction of transport force, for example.

During the processes of transport and loop length adjustment, it is advantageous to include a feature that allows this user to easily manipulate haul 105 and transport limb 106. A grasping loop 107 formed into the ends of haul 105 and transport limb 106 is shown. Grasping loop 107 is of a useful diameter and formed such that the user may place a finger or surgical device through the loop and apply a force to the respective limb without the diameter of grasping loop 107 changing. Methods to form grasping loop 107 may include but are not limited to a common bowline knot or Brummel lock-splice.

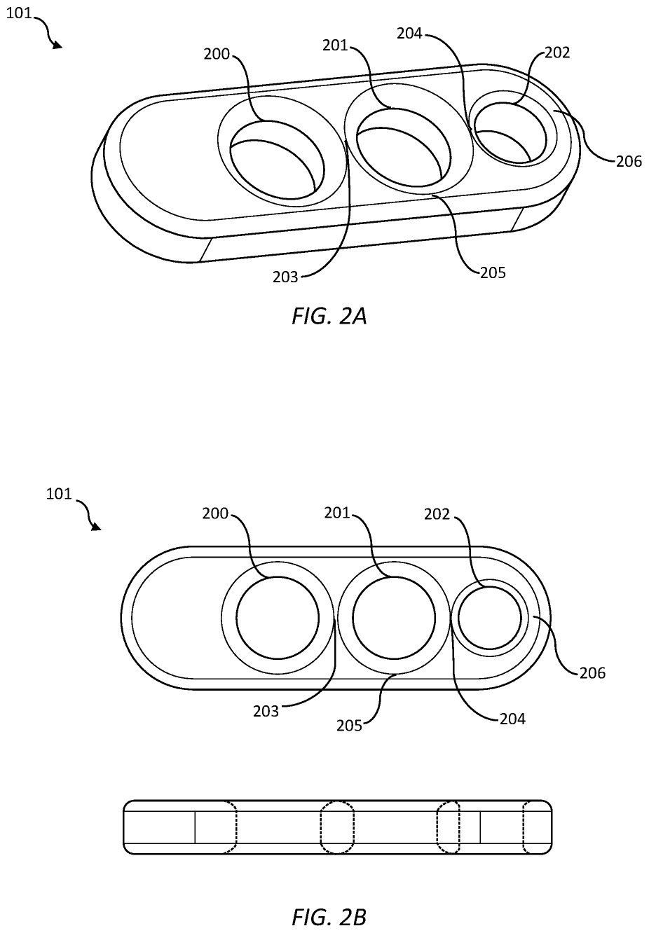

As shown in FIGS. 2A-2B, rigid member 101 has a long axis from the first end to the second end with a length which may range from approximately 8 millimeters to approximately 15 millimeters, and a short axis with a width which may range from approximately 3 millimeters to approximately 10 millimeters, and a length-width (L:W) ratio greater than 1. Rigid member 101 may have a thickness from a top side to a bottom side ranging from approximately 1 millimeter to approximately 6 millimeters. A plurality of apertures through rigid member 101 are formed for the passage of a fall during the adjustable loop reeving process. By forming multiple apertures, sheaves and beckets between the apertures are established which are used during the adjustable loop and knot forming process. Apertures formed generally nearest to the centerline of the rigid member can be referred to as inboard, whilst apertures formed generally closer to the ends of the part can be considered outboard for the purposes of this disclosure.

FIG. 2A illustrates a first inboard aperture 200 and a second inboard aperture 201 placed generally equidistant to the midpoint of the long axis of rigid member 101, creating a sheave 203 generally colinear with the short axis of rigid member 101. An outboard aperture 202 formed between aperture 201 and an end of rigid member 101 establishes a first becket 204 therebetween. FIG. 2B is a top and side view of rigid member 101 illustrating a second becket 205 formed by the span between aperture 201 and the perimeter of rigid member 101. A third becket 206 is also established spanning aperture 202 and the perimeter of rigid member 101. It should be noted that a becket or sheave may be formed between any two apertures or an aperture and the perimeter of the rigid member, and a sheave may also function as a becket.

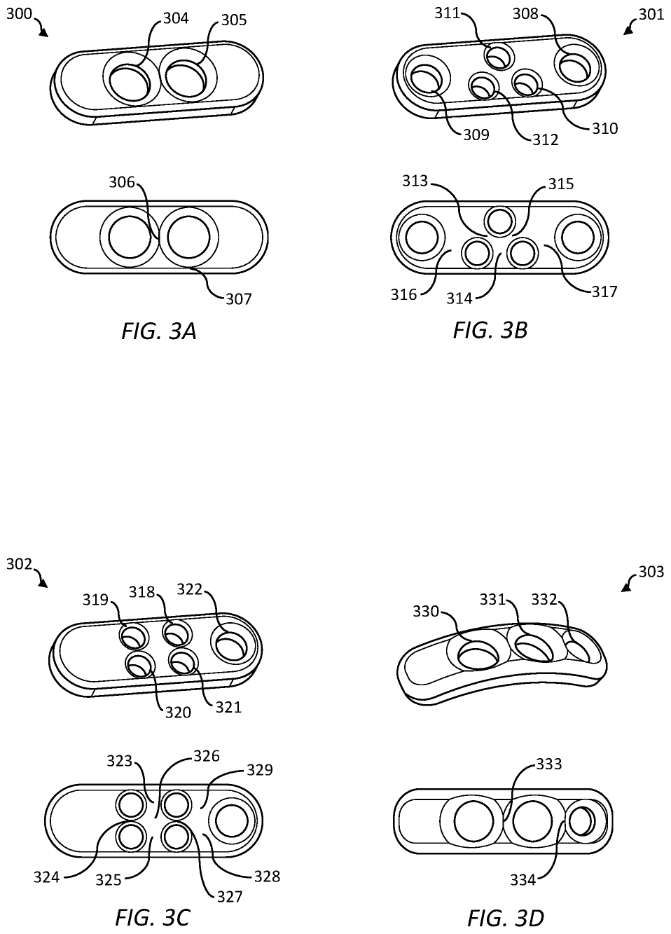

FIGS. 3A-3D illustrate several alternative and non-limiting embodiments of a rigid member, in accordance with the disclosure. The examples are intended to show that several aperture configurations are possible and should be appreciated in the application of the present invention. The aperture configuration and size may change with different applications of the device to provide control over which sections of a fall are in contact with each other during the adjustment process, and should not be limiting in the scope of the present invention.

In FIG. 3A, a rigid member 300, having similar proportions to rigid member 101, is shown formed with two apertures. A first inboard aperture 304 and a second inboard aperture 305 are formed establishing a sheave 306 between them. A becket 307 is formed between aperture 305 and the perimeter of rigid member 300.

FIG. 3B shows a rigid member 301, having similar proportions to rigid member 101, formed with five apertures. A first inboard aperture 310, a second inboard aperture 311, and a third inboard aperture 312 are formed in a triangular pattern with the center of the set of apertures located near the midpoint of the long and short axes of rigid member 301. A sheave 313 is then established spanning apertures 311 and 312, a sheave 314 is established spanning apertures 310 and 312, and a sheave 315 is formed spanning apertures 310 and 311. A first outboard aperture 308 is formed between the three inboard apertures and one end of rigid member 301 establishing a becket 316 spanning apertures 308 and 311, and a becket 317 spanning apertures 308 and 310. A second outboard aperture 309 is also formed, providing alternate becket locations and an auxiliary aperture if ancillary flexible members are attached for user manipulation.

FIG. 3C shows a rigid member 302, having similar proportions to rigid member 101, formed with five apertures. A first inboard aperture 318, a second inboard aperture 319, a third inboard aperture 320, and a fourth inboard aperture 321 are formed in a square array pattern with the center of the set of apertures located on both the long and short axes of rigid member 302. Thus, a sheave 323 is established spanning apertures 318 and 319, a sheave 324 is established spanning apertures 319 and 320, a sheave 325 is established spanning apertures 320 and 321, and a sheave 327 is established spanning apertures 318 and 321. A common sheave 326 is also established which spans apertures 319 and 321, and also spans apertures 320 and 322. An outboard aperture 322 is also shown, which establishes a becket 328 spanning apertures 321 and 322, and a becket 329 spanning apertures 318 and 322.

FIG. 3D shows an embodiment of a rigid member 303, having similar proportions to rigid member 101, having three apertures. Rigid member 303 is shown formed into a curved member with the radius of curvature generally congruent with the anatomical structure it is intended to oppose. A first inboard aperture 330 and a second inboard aperture 331 are formed establishing a sheave 333 therebetween. An outboard aperture 332 is formed between the first end of rigid member 303 and aperture 331, which establishes a becket 334 spanning apertures 332 and 331.

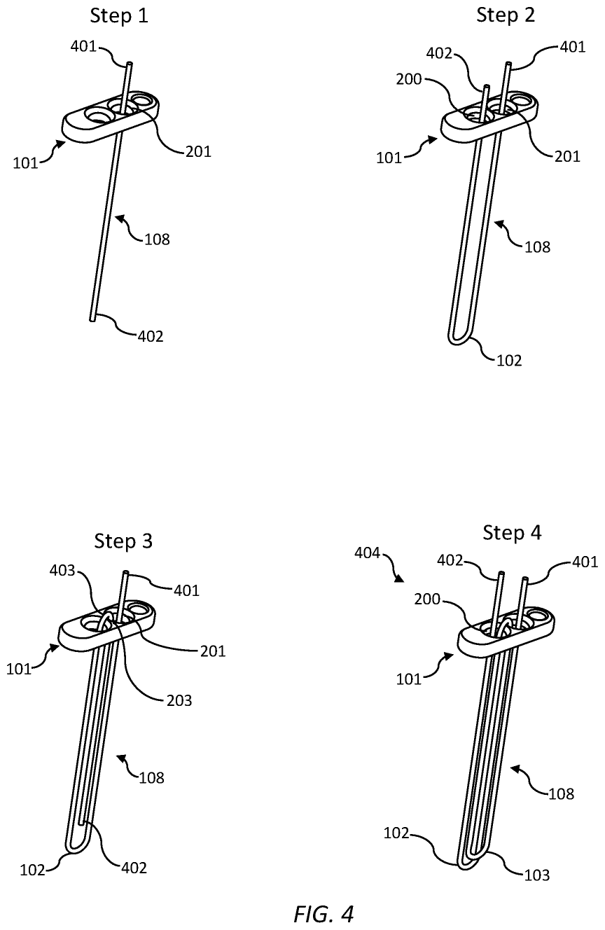

FIG. 4 illustrates exemplary steps for forming a tackle 404. The intent of this portion of the discussion is to illustrate a combined standing block and running pulley system using a rigid member and a flexible member with a fixed pulley represented in this embodiment by sheave 203, and the moveable pulley represented by adjustable loops 102 and 103. Steps one through four in FIG. 4 illustrate one method of creating such a tackle. One skilled in the art would recognize that there are several methods to arrive at the configuration of the embodiment described below, and the method to arrive at such configuration should not be limiting to the scope of the present invention. Step one shows fall 108, with a first end 401 passed from the bottom side of rigid member 101 through aperture 201 extending from the top side of rigid member 101. Step two shows a second end 402 transported through aperture 200 from the bottom side of rigid member 101 to the top side, thereby forming adjustable loop 102 with a distance to the bottom side of rigid member 101 ranging from approximately 5 centimeters to 40 centimeters. Step three illustrates end 402 being transported through aperture 201 from the top side of rigid member 101 passing over sheave 203 and extending from bottom side of rigid member 101 toward adjustable loop 102, thereby establishing a pulley loop 403. Step four shows end 402 passed through aperture 200 from the bottom side of rigid member 101 extending from top side a second time, which establishes adjustable loop 103 having similar length to adjustable loop 102.

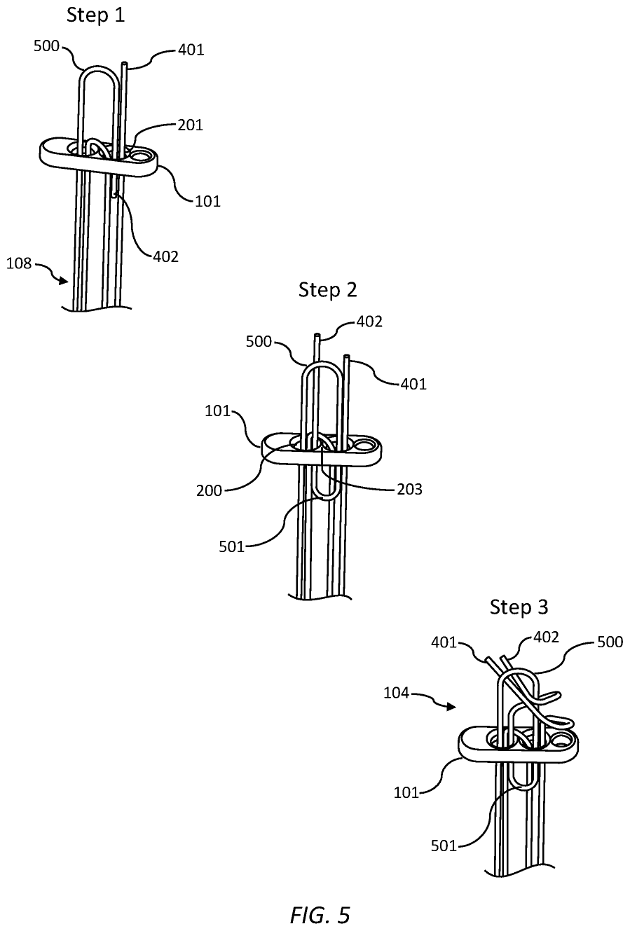

FIG. 5 illustrates exemplary steps for forming sliding self-locking knot 104. In this embodiment the starting configuration of the knot-tying process begins with tackle 404. Step one shows end 402 of fall 108 being transported through aperture 201 from the top side of rigid member 101 extending from the bottom side while leaving a locking loop 500 extending from the top side of rigid member 101. Step two illustrates end 402 passed through aperture 200 from the bottom side of rigid member 101 extending from the top side establishing an end fixing loop 501 engaging sheave 203. It should be noted that in this example, sheave 203 also functions as a becket. Ends 401 and 402 are now both extending from the top side of rigid member 101. Step three shows both ends 401 and 402 passed together though locking loop 500 from the near side to the far side. Knot 104 can then be consolidated to enable the adjustable and self-locking features heretofore described with end 401 functional as transport limb 106 and end 402 functional as haul 105.

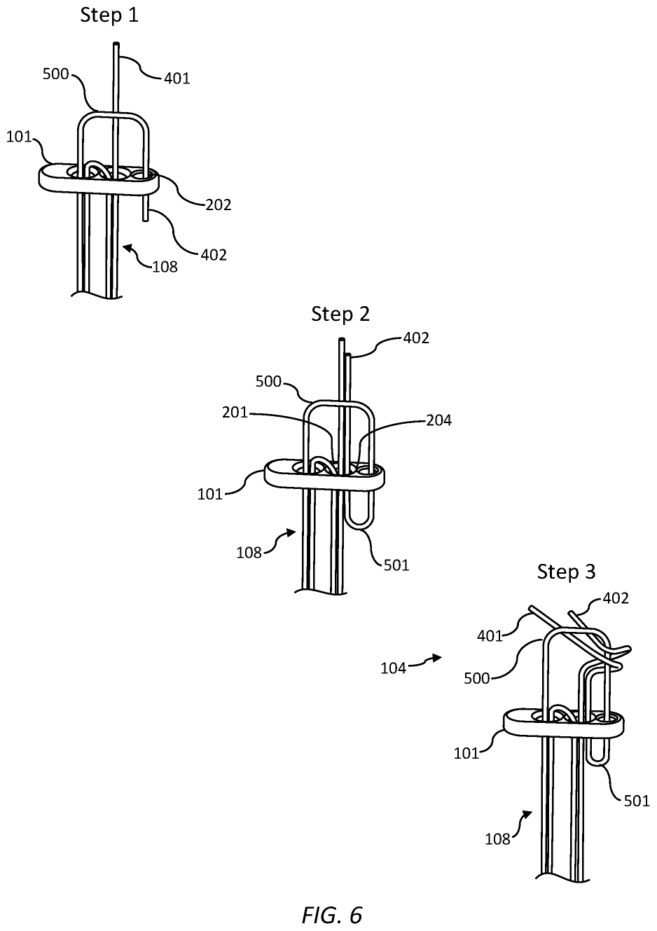

FIG. 6 illustrates an alternative embodiment of the exemplary steps for forming knot 104. In this embodiment the starting configuration of the knot-tying process begins with tackle 404. Step one shows end 402 passed through aperture 202 from the top side of rigid member 101 extending from bottom side and forming locking loop 500 extending from the top side of rigid member 101. Step two shows end 402 subsequently passed through aperture 201 from the bottom side of rigid member 101 extending from the top side and establishing end fixing loop 501 engaging with becket 204. End 402 arrives on the same side of locking loop 500 as end 401, toward the far side of rigid member 101 with respect to locking loop 500 in this example. Step three illustrates both ends 401 and 402 passed through locking loop 500 from the near side to the far side. Knot 104 can then be consolidated to enable the adjustable and self-locking features heretofore described with end 401 functional as transport limb 106 and end 402 functional as haul 105.

It should be noted that the passage of an end through an aperture to engage around a becket, referred to as an end fixing loop establishes the fixed end of the fall of a pulley system as well as forming a locking loop. One skilled in the art would recognize that there are several methods to arrive at the configuration of the embodiments described in FIGS. 5 and 6, and the method to arrive at such configurations or the choice of apertures or beckets used to establish a locking loop or an end fixing loop should not be limiting to the scope of the present invention.

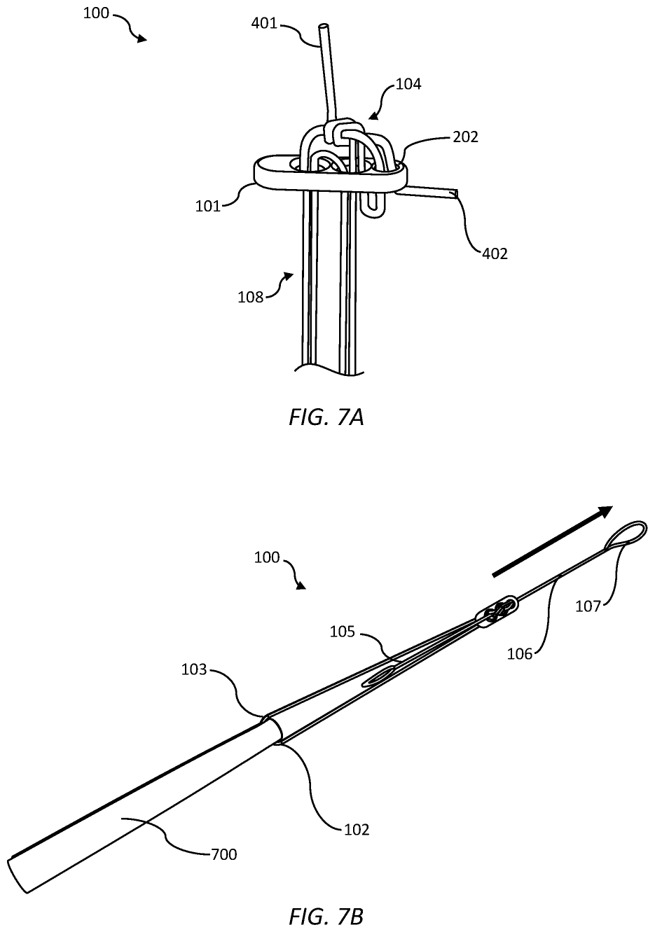

Once knot 104 has been consolidated, both transport haul 105 and transport limb 106 are generally in the middle of the long axis of the rigid member and on the top side in the embodiments presented. Transport of the construct through tissue can be achieved by applying a force to transport limb 106. The rigid member will have a natural tendency to orient its top side normal to the direction of the force applied to the transport limb. In certain cases where a bone tunnel through which the device is passed is smaller in diameter than the length of the rigid member, it would be advantageous for the rigid member to be oriented such that its long axis is generally parallel to the transport limb. FIG. 7A illustrates an exemplary step for enabling the control of the orientation of rigid member 101 when suspensory fixation device 100 is transported through a bone tunnel or narrow passage. End 402 is passed through aperture 202 from the top side of rigid member 101 and extending from the bottom side. It should be noted that the choice of outboard aperture used in this step would produce a variety of possible orientation which may be advantageous to the user and should not be limiting to the scope of the present invention.

FIG. 7B is a perspective view of suspensory fixation device 100 integrated with a tissue graft 700 and oriented for transportation through a tunnel or passage. When a pulling force is applied to transport limb 106, rigid member 101 is shown to orient itself with its long axis generally parallel to the direction of transport. The orientation achieved will allow rigid members with a length-width ratio greater than 1 to be transported through passages formed in a bone with diameters substantially close to the width of the rigid body.

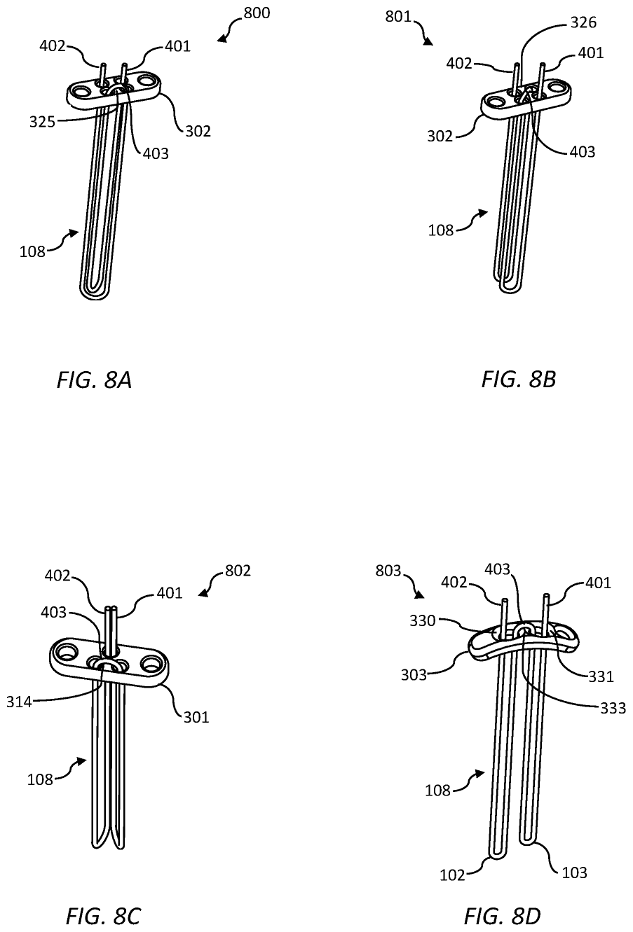

FIGS. 8A-8D illustrate alternative embodiments of tackle 404. FIG. 8A illustrates a tackle 800 formed from passing ends 401 and 402 of fall 108 through apertures in rigid member 302 to arrive at the configuration shown, where pulley loop 403 passes over sheave 325. A configuration of this type results in that for each passage though rigid member 302, the ends of fall 108 are contained in separate apertures. FIG. 8B illustrates a tackle 801 formed by passing ends 401 and 402 of fall 108 through apertures in rigid member 302 to arrive at the configuration shown, where pulley loop 403 passes over sheave 326. A configuration of this type results in that for each passage through rigid member 302, the ends of fall 108 are contained in separate apertures. FIG. 8C illustrates a tackle 802 formed by passing ends 401 and 402 of fall 108 through apertures in rigid member 301 to arrive at the configuration shown, where pulley loop 403 passes over sheave 314. A configuration of this type results in ends 401 and 402 both extending through aperture 311. FIG. 8D illustrates a tackle 803 formed by passing ends 401 and 402 of fall 108 through apertures in rigid member 303. End 401 is first passed through aperture 331 from the top side of rigid member 303 extending from the bottom side. End 401 is then passed through aperture 331 from the bottom side of rigid member 303 extending from the top side and establishing adjustable loop 103. End 402 is then passed through aperture 330 from the top side of rigid member 303 and extending from the bottom side establishing pulley loop 403 over sheave 333. End 402 is then passed through aperture 330 from the bottom side of rigid member 303 from the bottom side to the top side establishing adjustable loop 102, having similar length to adjustable loop 103.

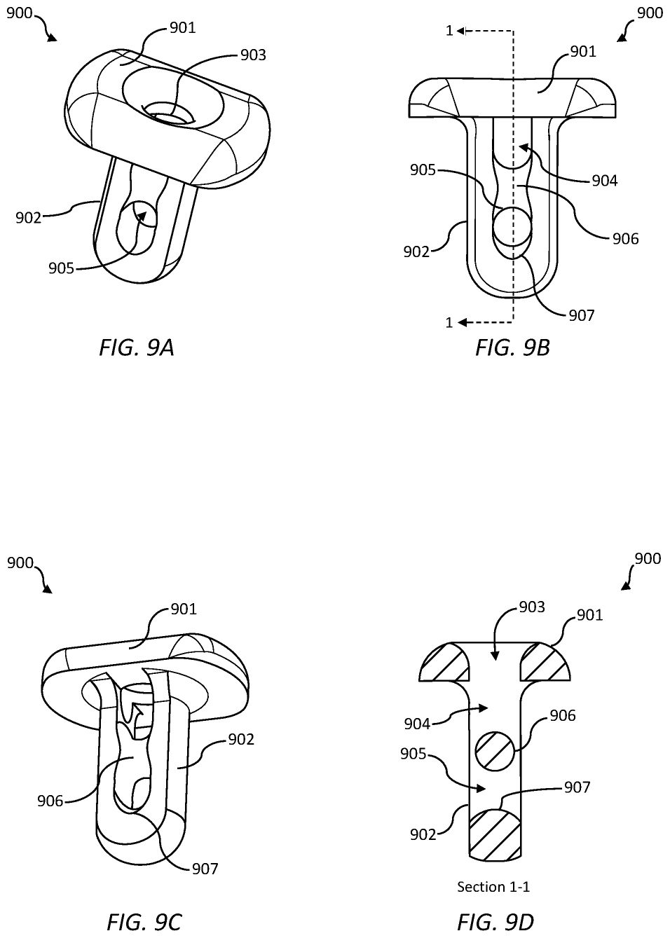

FIGS. 9A-9D are perspective, side, and section views of a rigid member 900, according to an embodiment of the present invention. FIG. 9A is a perspective view of rigid member 900 having a generally flat upper body 901 having a top and bottom and connected to a centralizing post 902, which has a first side and a second side, extending perpendicularly to a distance approximately 3 millimeters to approximately 15 millimeters from the center of the bottom of upper body 901. Upper body 901 may be generally circular, with a diameter ranging from approximately 6 millimeters to approximately 15 millimeters, or generally oblong, with a long axis dimension ranging up to 15 millimeters and an L:W ratio less than 3:1. Upper body 901 has a central, axial aperture 903 which extends colinearly to and partially into centralizing post 902. FIG. 9B illustrates a connecting aperture 904, formed from side 1 to side 2 of centralizing post 902, connecting with axial aperture 903. This connection forms a void recessed from the top surface of upper body 901 as well as a path for passing an end of fall 108 such that it may enter connecting aperture 904 from either side 1 or side 2 of centralizing post 902 and exit through axial aperture 903. An aperture 905 is formed through centralizing post 902 from the first side to the second side, establishing a becket 906 between connecting aperture 904 and aperture 905, and also establishes a sheave 907 between aperture 905 and the end of centralizing post 902. Upper body 901 should have at least one dimension sufficiently larger than the maximum width or diameter of centralizing post 902 such that a shelf is created to sufficiently span a hole in a plate or bone in which centralizing post 902 resides, to provide fixation post for a particular surgical indication. FIG. 9D is a section view of rigid member 900, where the connection between axial aperture 903 and connecting aperture 904 may be appreciated.

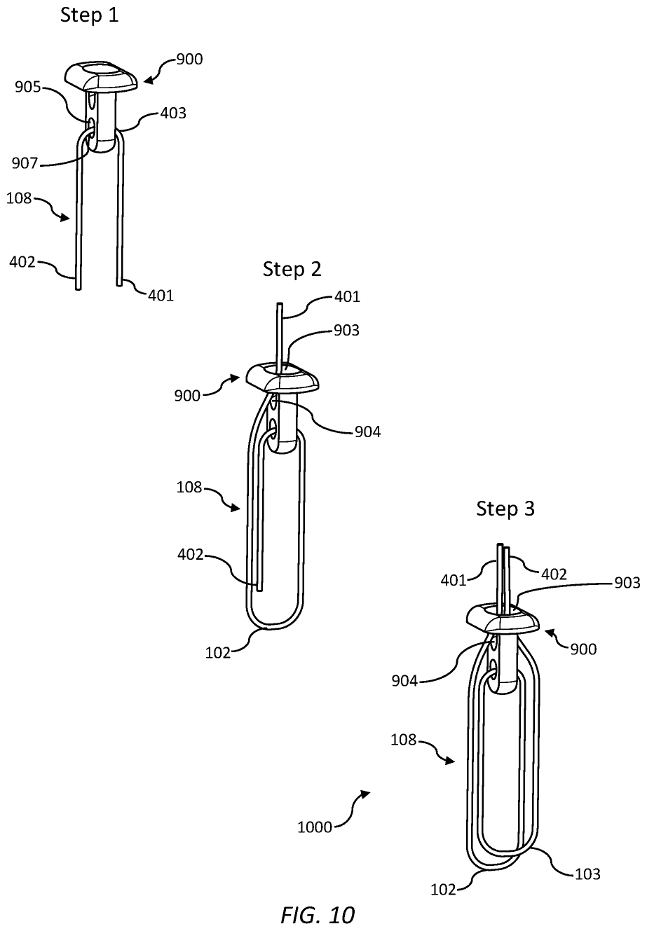

FIG. 10 illustrates exemplary steps for creating an alternative embodiment of the tackles described heretofore. A tackle 1000, comprising of rigid member 900 and fall 108 is shown. Step one illustrates end 401 passed through aperture 905 from the first side of centralizing post 902 to the second side, establishing pulley loop 403 sliding on sheave 907. Step two shows end 401 passed into connecting aperture 904 from the first side of centralizing post 902, and exiting axial aperture 903, thereby establishing adjustable loop 102. Step three shows end 402 passed into connecting aperture 904 from the second side of centralizing post 902 and exiting axial aperture 903, establishing adjustable loop 103 having a length generally similar to adjustable loop 102.

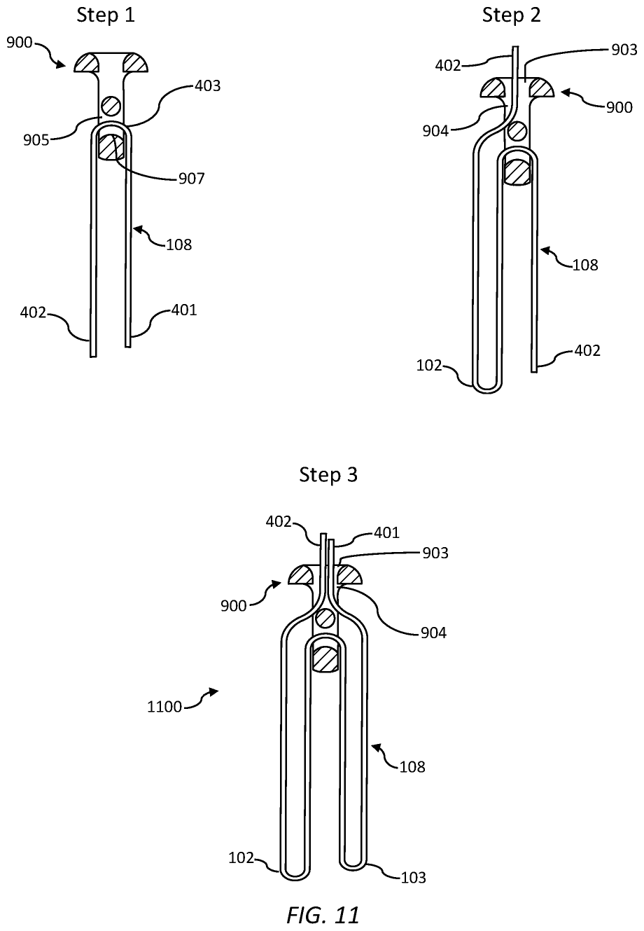

In another embodiment, FIG. 11 illustrates the exemplary steps for forming a tackle 1100 comprising of rigid member 900 and fall 108. In this example, Step one shows end 401 passed through aperture 905 from the first side of centralizing post 902 to the second side, establishing pulley loop 403 sliding on sheave 907. Step two shows end 402 subsequently passed into connecting aperture 904 from the first side of centralizing post 902 and exiting axial aperture 903, establishing adjustable loop 102. Step three shows end 401 passed into connecting aperture 904 from the second side of centralizing post 902 and exiting axial aperture 903, establishing adjustable loop 103 having generally similar length to adjustable loop 102.

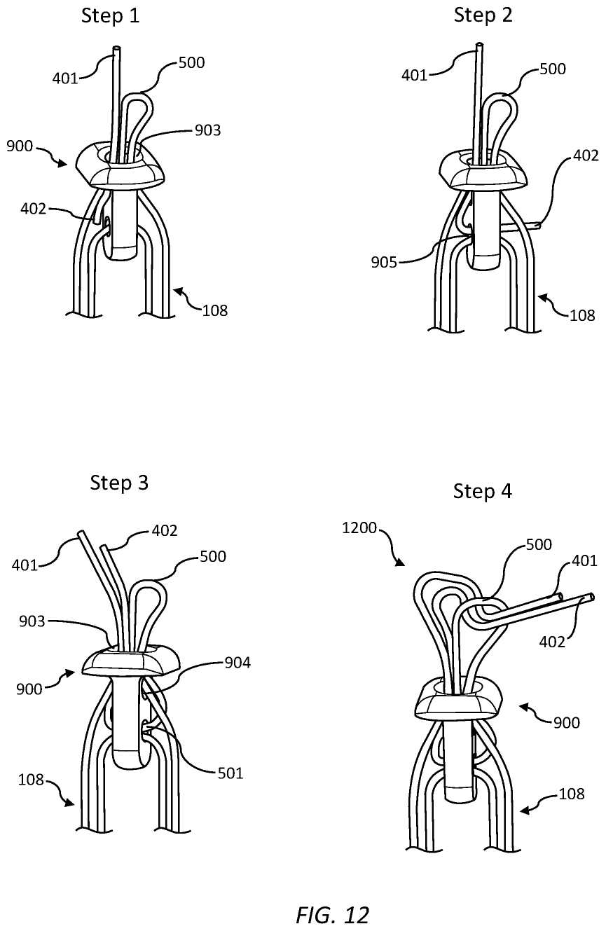

FIG. 12 illustrates exemplary steps for creating a sliding, self-locking knot 1200 in tackle 1000 according to an embodiment of the present invention. Step one illustrates end 402 passed into axial aperture 903 from the top side of upper body 901, exiting connecting aperture 904 to arrive on the first side of centralizing post 902, establishing locking loop 500 extending from the top surface of upper body 901. Step two shows end 402 passed through aperture 905 from the first side of centralizing post 902 and exiting the second side. Step three shows end 402 passed into connecting aperture 904 from the second side of centralizing post 902 and exiting axial aperture 903, establishing end fixing loop 501 engaging becket 906. Step four shows ends 401 and 402 passed together through locking loop 500. Knot 1200 can then be consolidated thereby enabling the sliding and self-locking features as previously described.

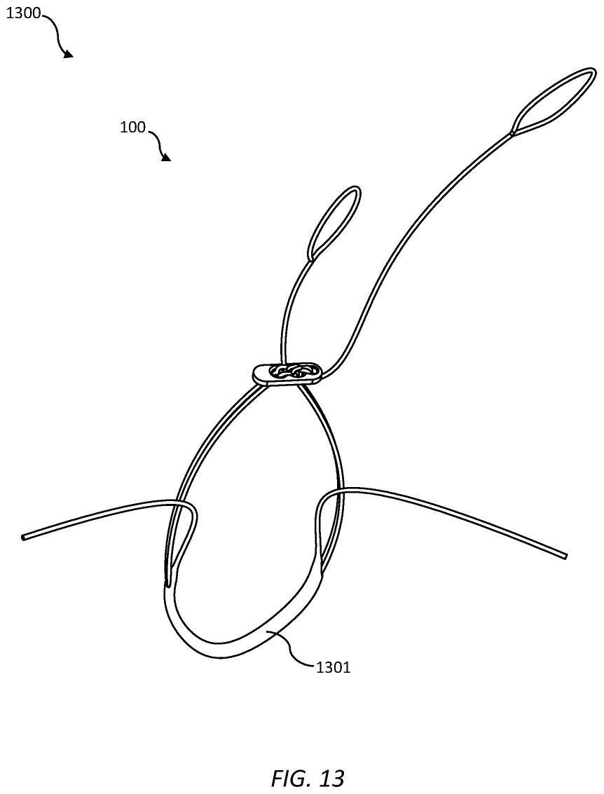

FIG. 13 is a perspective view of a suspensory fixation device 1300, comprising of suspensory fixation device 100 integrated with a graft support member 1301, according to an embodiment of the present invention. Graft support member 1301 may be a flexible cable or suture which may be a hollow monofilament or manufactured from a plurality of biologically-compatible fibers braided into a cable having at least a portion of the cable comprising a hollow cavity, and may or may not be the same material used in the manufacture of fall 108. Non-limiting examples of permanent materials for graft support member 1301 are ultra-high molecular weight polyethylene (UHMWPE), polyester, polypropylene, carbon fiber, metal wires, or silk. Non-limiting examples of resorbable materials for graft support member 1301 having limited lifespan in the body are polylactic acid (PLA), polyglycolic acid (PGA), and polygalactin. Graft support member 1301 may be manufactured in manner such that apertures may be opened between the braided fibers or into the wall of the hollow monofilament, allowing access for at least one other flexible member to be passed into the hollow portion through the first aperture, passed through the hollow portion, and exit the hollow portion through the second aperture.

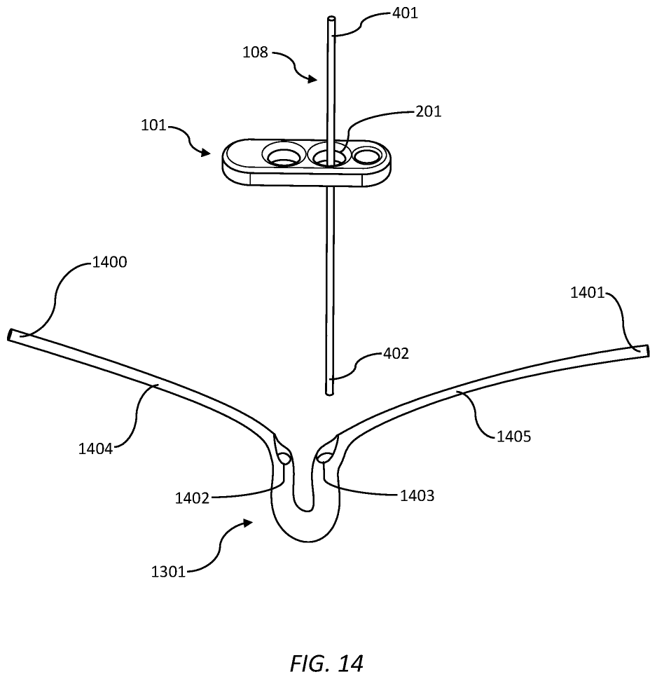

FIG. 14 illustrates the components of suspensory fixation device 1300, comprising of rigid member 101, fall 108, and graft support member 1301 oriented for assembly, according to one embodiment of the present invention. In this example, graft support member 1301 is shown having a first end 1400 and a second end 1401 with a length ranging from approximately 10 centimeters to approximately 50 centimeters. Graft support member 1301 has a first aperture 1402 formed between end 1400 and end 1401, and a second aperture 1403 located between aperture 1402 and end 1401. A first limb 1404 is defined as the length of graft support member 1301 between end 1400 and aperture 1402. A second limb 1405 is then defined as the length of graft support member 1301 between aperture 1403 and end 1401. The distance between apertures 1402 and 1403 may range from approximately 10 millimeters to approximately 30 millimeters. End 401 is passed through aperture 201 from the top side of rigid member 101 and extends from the bottom side as shown.

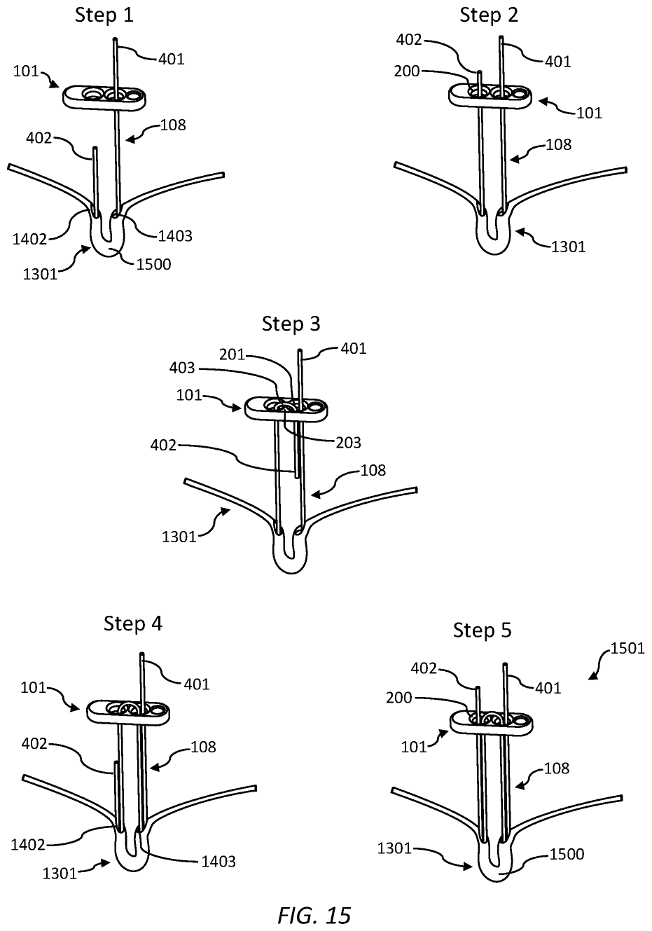

FIG. 15 illustrates exemplary steps for forming a tackle 1501, which is a precursor to suspensory fixation device 1300. Step one illustrates end 402 of fall 108 first passed through aperture 1403 to enter the annulus of graft support member 1301 and subsequently exiting through aperture 1402, thereby establishing a saddle 1500. Step two illustrates end 402 passing through aperture 200 from the bottom side of rigid member 101 and extending from the top side. Step three shows end 402 passed through aperture 201 from the top side of rigid member 101 extending from the bottom side, establishing pulley loop 403 engaging sheave 203. Step four shows end 402 being passed through aperture 1403 for a second time and exiting aperture 1402. Step five shows end 402 passed through aperture 200 from the bottom side of rigid member 101 and extending from the top side, completing the steps for forming tackle 1501. Knot 104 and grasping loops 107 (not shown) may then be tied as previously described to complete the formation of suspensory fixation device 1300.

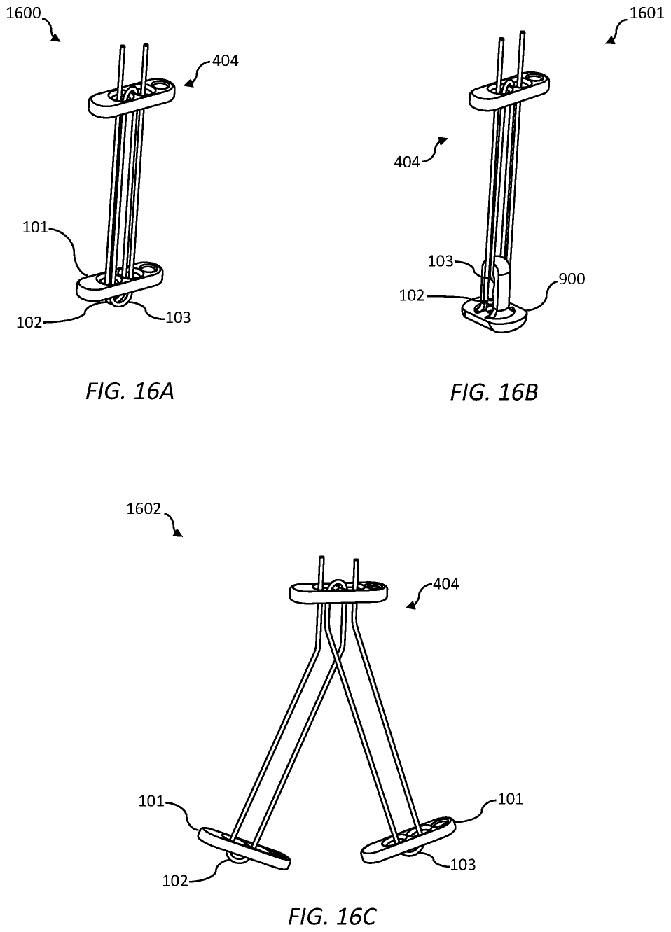

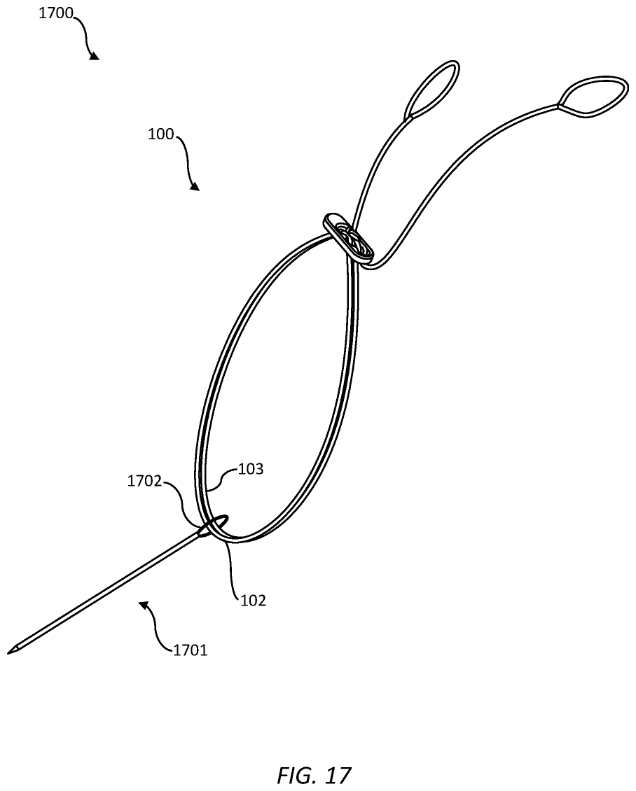

FIGS. 16A-16C illustrate further examples of tackles integrated with additional rigid members. In FIG. 16A, one example of a tackle 1600, comprising of tackle 404 and a second rigid member 101 located in proximity of adjustable loops 102 and 103 is shown. The steps for forming this example are similar to the methods described heretofore and may be referenced. FIG. 16B shows a further example of a tackle 1601. In this embodiment, and is formed using tackle 404, and rigid member 900 configured using the methods previously described. In this example, adjustable loops 102 and 103 are passed through separate apertures in centralizing post 902, such that the movable pulley established in this example has its load shared between becket 906 which functions as a sheave in this example and sheave 907. FIG. 16C illustrates a tackle 1602 comprising tackle 404 with a second rigid member 101 integrated onto adjustable loop 102 and a third rigid member 101 integrated onto adjustable loop 103.

FIG. 17 is a perspective view of a suspensory fixation device 1700. This embodiment contains components previously introduced in FIG. 1. In this example, a surgical needle 1701 having an eyelet 1702 is integrated onto the adjustable loops 102 and 103 of suspensory fixation device 100. Using a process similar to the steps described in FIG. 4, end 402 is passed through eyelet 1702 during the formation of adjustable loop 102, and then again passed through eyelet 1702 during the formation of adjustable loop 103. Eyelet 1702 may have a diameter large enough to allow for free movement about adjustable loops 102 and 103 and have sufficient strength to manipulate the adjustable loops through tissue. Should the user need to transport adjustable loops 102 and 103 together through tissue, needle 1701 would be operable in length and rigidity to facilitate transport by providing the means of opening the aperture in the tissue and manipulating the adjustable loops through that aperture in one step.

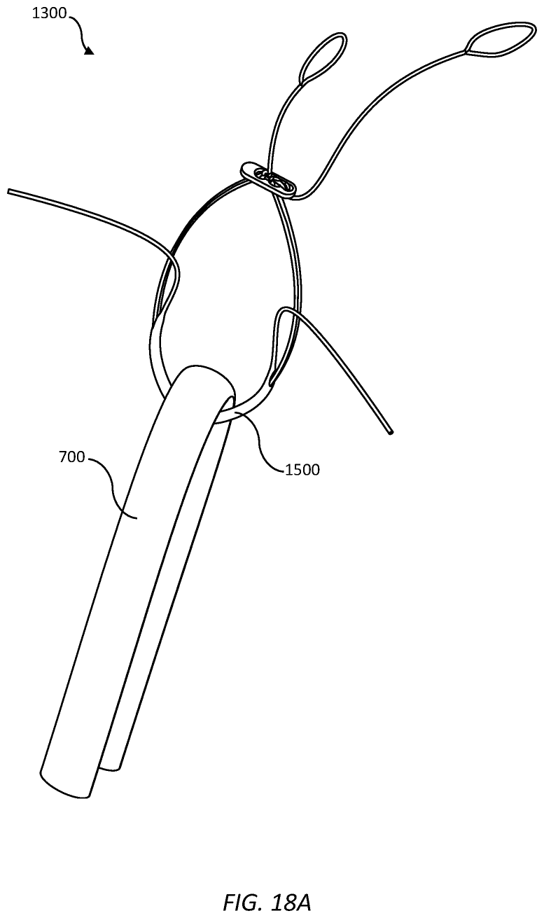

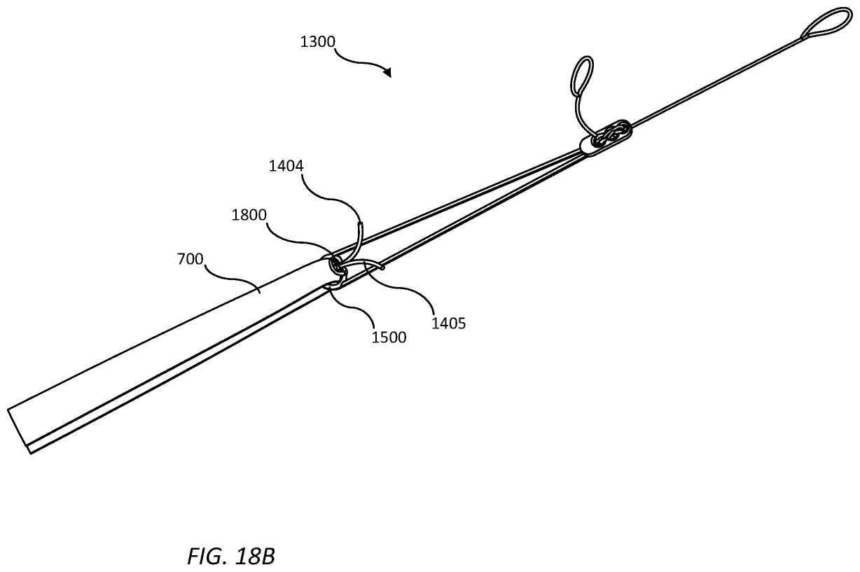

FIGS. 18A-18B illustrate one use of suspensory fixation device 1300. FIG. 18A illustrates tissue graft 700 being passed over saddle 1500 and through adjustable loops 102 and 103, and then folded back onto itself. FIG. 18B illustrates limbs 1404 and 1405 tied into a suitable non-sliding surgical knot 1800, thereby securing tissue graft 700 to graft support member 1301. In this configuration, saddle 1500 will isolate adjustable loops 102 and 103 from tissue graft 700 to prevent damage from sliding friction to the graft during the adjustment process.

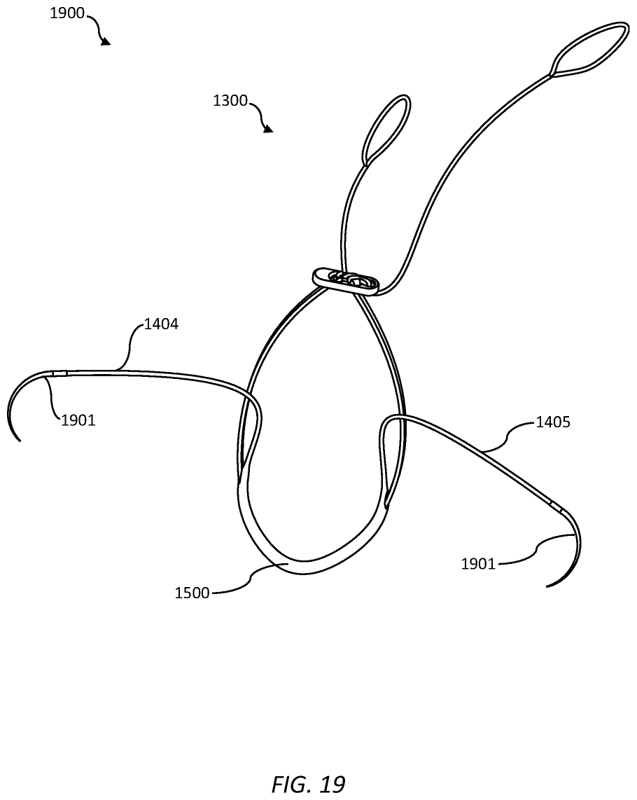

FIG. 19 is a perspective view of an embodiment of a suspensory fixation device 1900 for improved security of the implant to a tissue graft comprising suspensory fixation device 1300 having a first curved needle 1901 affixed to end 1400, and a second curved needle 1901 affixed to end 1401. Several styles of curved and straight needles are commercially available. The user may select the needle gage and radius of curvature to match the performance required for various tissues or surgical techniques. It should be noted that the needle styles affixed to ends 1400 and 1401 may be different or similar and the method of affixing the needles should not be limiting to the scope of the present invention.

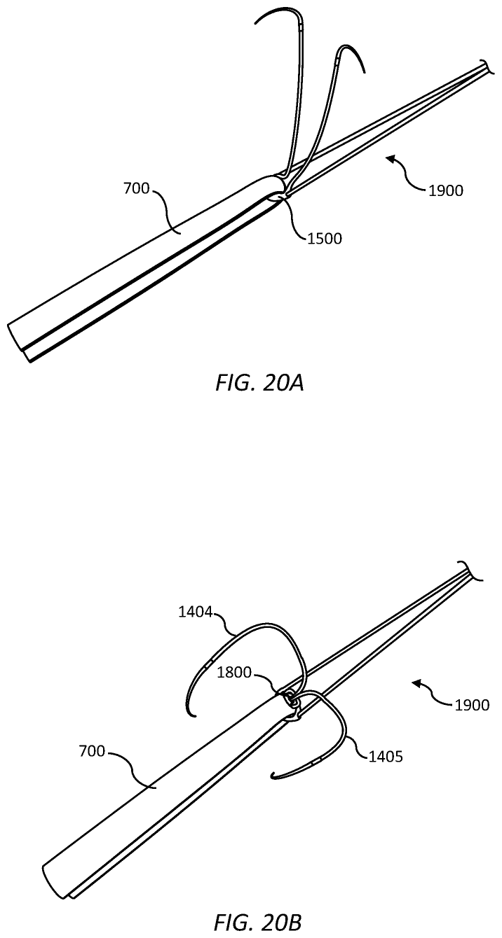

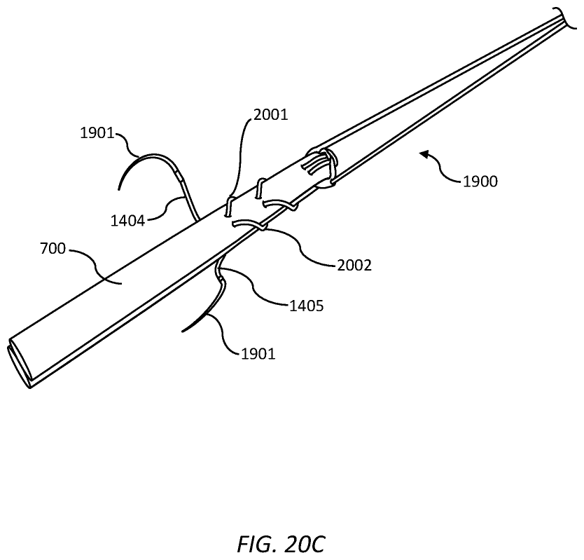

FIGS. 20A-20C illustrate one use of suspensory fixation device 1900 according to an embodiment of the present invention. FIG. 20A illustrates tissue graft 700 positioned over saddle 1500 in similar fashion previously described in FIG. 18A. FIG. 20B shows limbs 1404 and 1405 tied into knot 1800 securing saddle 1500 at a fixed location on tissue graft 700 similarly illustrated in FIG. 18B. Should the user prefer or require additional fixation to suspensory fixation device 1900, the user may first use needle 1901 to whip stitch limb 1404 into tissue graft 700. Subsequently, limb 1405 may be whip stitched into tissue graft 700 in similar fashion.

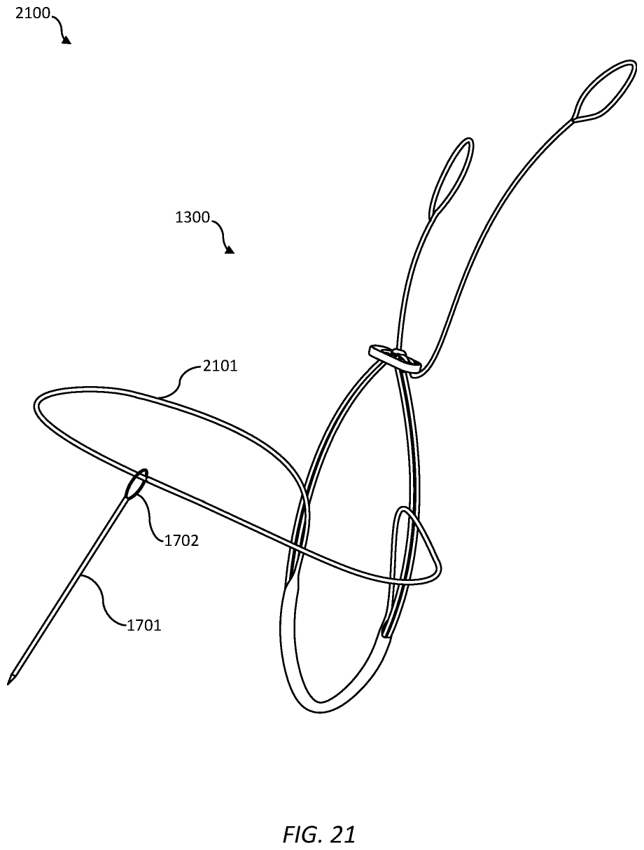

FIG. 21 is a perspective view of a suspensory fixation device 2100. In this example, ends 1400 and 1401 (not shown) are spliced together to form a whipstitch loop 2101 with needle 1701 moveable on whipstitch loop 2101 by its eyelet 1702.

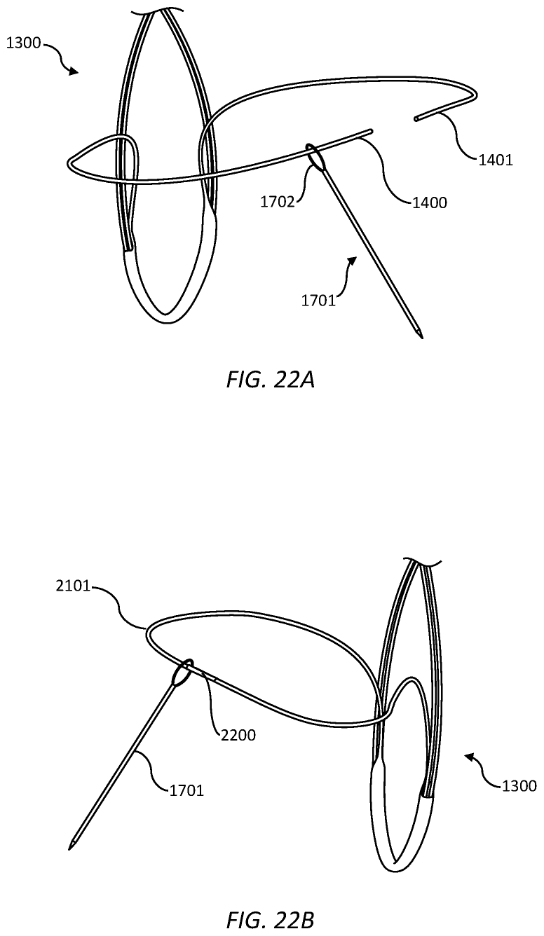

FIGS. 22A-22B illustrate exemplary steps for forming whipstitch loop 2101 and affixing needle 1701 thereto, according to an embodiment of the present invention. FIG. 22A shows end 1400 being passed through eyelet 1702. FIG. 22B shows a splice 2200 created to join ends 1400 and 1401 to create whipstitch loop 2101 on which needle 1701 may freely slide. A skilled artisan will recognize that various splicing or knot tying methods have been disclosed to form end-to-end splices in hollow cables, and the method of joining ends 1400 and 1401 should not be limiting to the scope of the present invention.

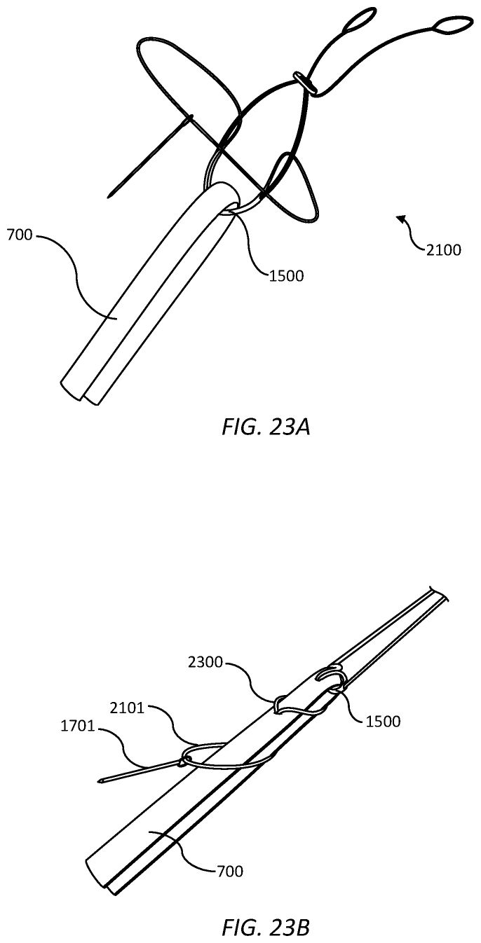

FIGS. 23A-23B illustrate one use of suspensory fixation device 2100 according to and embodiment of the present invention. In this example, FIG. 23A shows tissue graft 700 positioned over saddle 1500. FIG. 23B shows a stitch 2300 created by passing whipstitch loop 2101 through both legs of tissue graft 700 using needle 1701 as a transport mechanism and then bringing whipstitch loop 2101 over the ends of tissue graft 700. One skilled in the art would appreciate the continued iteration of these steps may be described as fabricating a “baseball style” stitch.

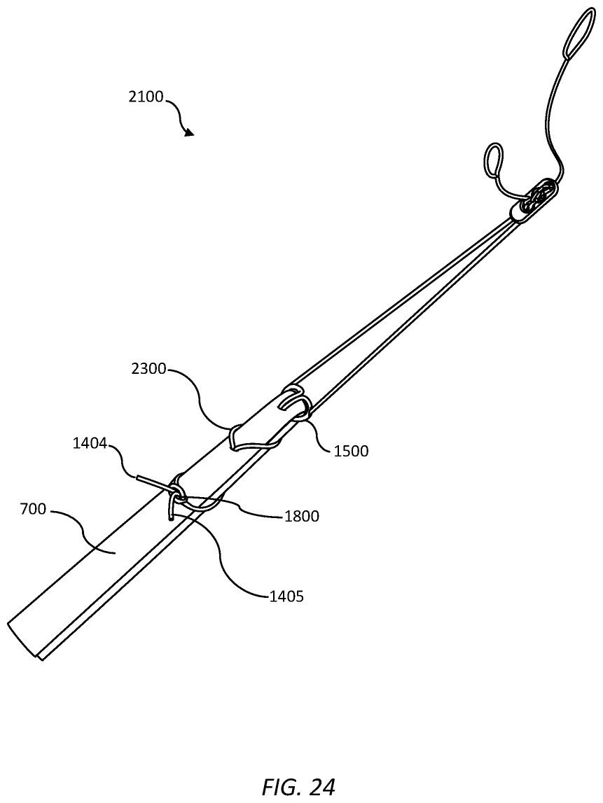

FIG. 24 illustrates suspensory fixation device 2100 secured to tissue graft 700. In this example, tissue graft 700 is shown whipstiched to suspensory fixation device 2100 as described heretofore. Limbs 1404 and 1405 are then tied to form knot 1800 and then trimmed. Stitching limbs 1404 and 1405 to tissue graft 700 fixes its position onto saddle 1500 causing the legs of tissue graft 700 to be isolated from each other enabling the legs to function as separate and independent graft bundles with different amounts of tension to be applied to each leg, most notably in double-bundle style ligament reconstruction procedures.

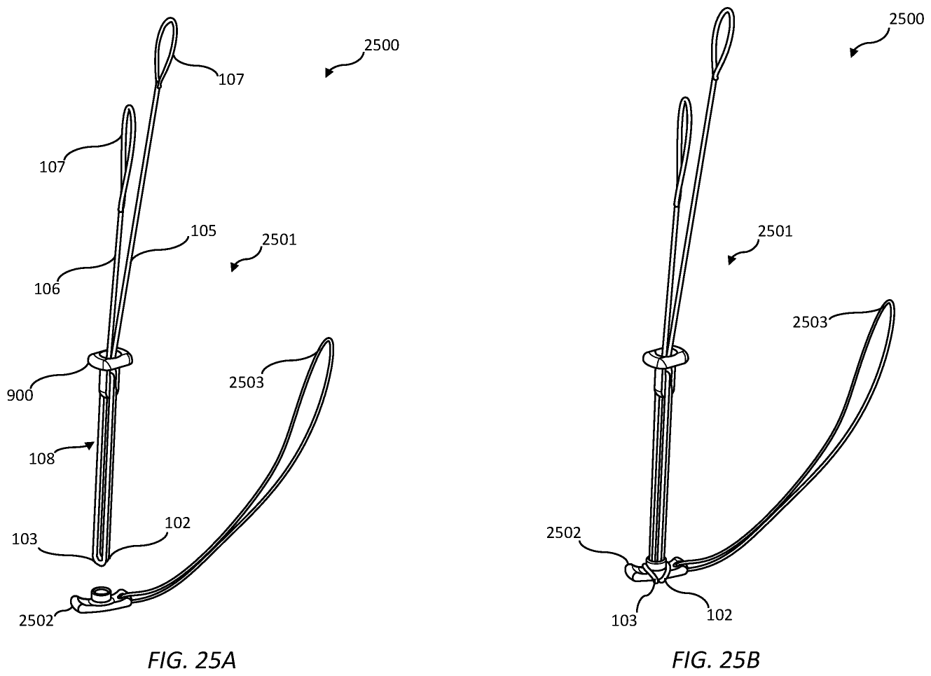

FIGS. 25A-25B illustrate the components of a suspensory fixation system 2500 comprising of an adjustable suspensory fixation device 2501 and a rigid member 2502, according to an embodiment of the present invention. Suspensory fixation device 2501 comprises rigid member 900 with fall 108 woven through its apertures, as previously described by the exemplary steps illustrated in FIG. 10, to form adjustable loops 102 and 103 with self-locking adjustable knot 1200 (not shown) tied as described by the exemplary steps illustrated in FIG. 12. Also included in suspensory fixation device 2501 are haul 105 and transport limb 106, each having grasping loop 107 woven into their respective ends. FIG. 25A illustrates rigid member 2502 unattached from suspensory fixation device 2501 and having a retrieval suture 2503 coupled to provide a method of manipulation demonstrating its design as a two-piece system intended for the user to assemble during the surgical procedure. FIG. 25B is a perspective view of suspensory fixation system 2500 in its assembled form.

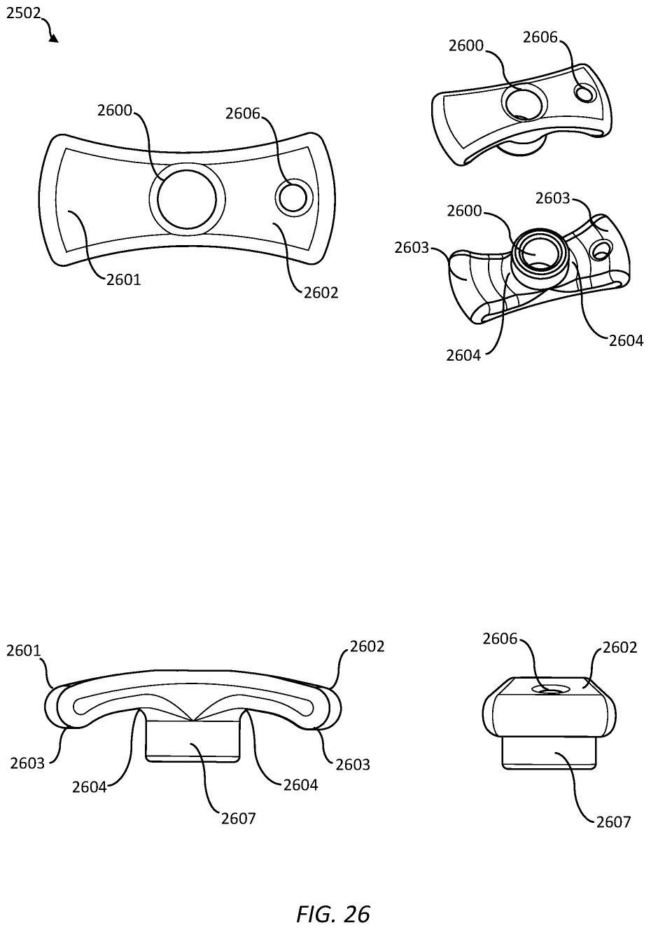

FIG. 26 illustrates various views of rigid member 2502 having a tubular center body 2607 with a proximal end and a distal end. Extending radially from the proximal end are a first lobe 2601 and a second lobe 2602 arranged perpendicular to the central axis of center body 2607 and arranged opposingly. Lobes 2601 and 2602 have a proximal side and a distal side and are curved toward the distal end of center body 2607 to form a bone-contacting pad 2603 at the end of each lobe. An impingement relief 2604 is shown as the recess created to enable the free movement of fall 108 around a sheave during the adjustment process. An aperture 2600 is formed through the centerline of center body 2607 with an inner diameter large enough such that both adjustable loops 102 and 103 (not shown) can be transported therethrough from the distal side to the proximal side together. In this example, an auxiliary aperture 2606 is formed in lobe 2602 for the purpose of attaching flexible cables or sutures for manipulation or retrieval of rigid member 2502. Rigid member 2502 may be manufactured from biologically compatible solid materials with suitable strength for a particular indication. Non-limiting examples of such materials would be titanium, alloys of titanium, alloys of stainless steel, polyether ether ketone (PEEK), or other fiber reinforced thermoset or thermoplastic composites.

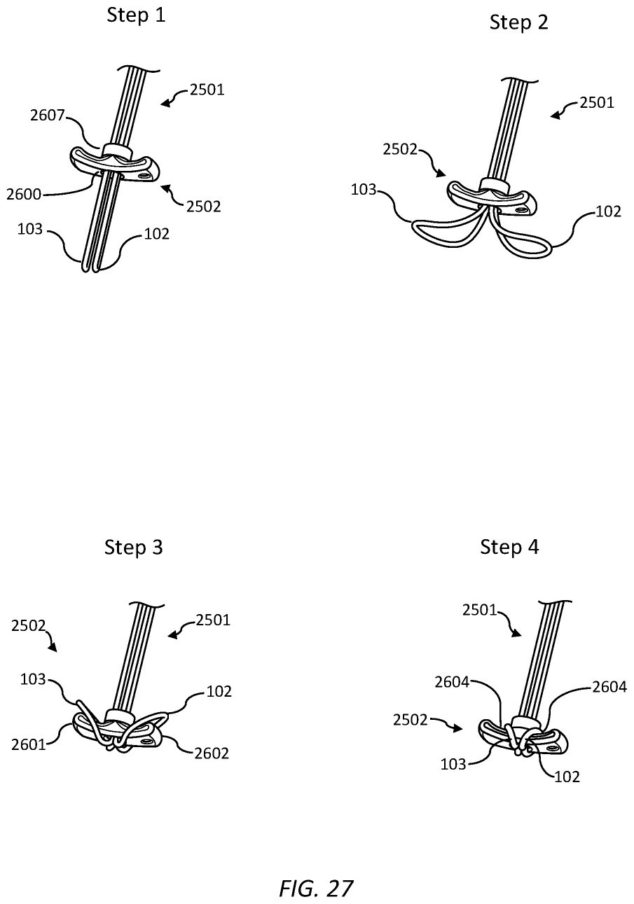

FIG. 27 illustrates the exemplary stems for assembling rigid member 2502 onto the adjustable loops of suspensory fixation device 2501, in accordance with at least one embodiment disclosed. Steps one illustrates both adjustable loops 102 and 103 passed through aperture 2600 from the distal side of center body 2607 and extending from the proximal side. Step two shows adjustable loops 102 and 103 separated from each other and the loops extending from the proximal side of rigid member 2502 each having large enough diameters to extend over the outside edges of lobes 2601 and 2602. Step three illustrates adjustable loop 102 placed over the outside edge of lobe 2602 to arrive on the distal side. Similarly, adjustable loop 103 is placed over the outside edge of lobe 2601. Step four illustrates that reducing the diameter of the adjustable loops will consolidate adjustable loops 102 and 103 their respective impingement relief 2604, and will not travel back to the proximal side of the lobe unless manipulated by the user.

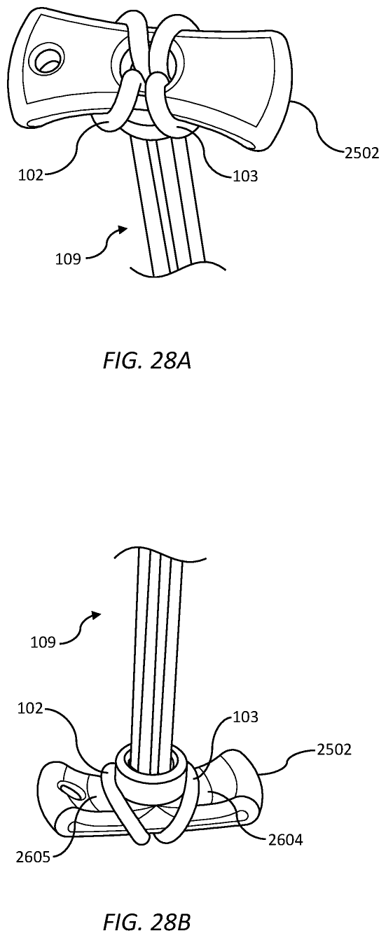

FIGS. 28A-28B are close-up, perspective views of adjustable loops 102 and 103 assembled onto rigid member 2502 in to illustrate their configuration on their respective lobes. During the loop length adjustment process, the user will apply a force to haul 105 (not shown) causing the pulley system to reduce the diameters of adjustable loops 102 and 103, thereby causing rigid member 2502 to translate toward rigid member 900 (not shown). In this example, the root of each lobe of where it connects to functions as a sheave on which each corresponding loop slides.

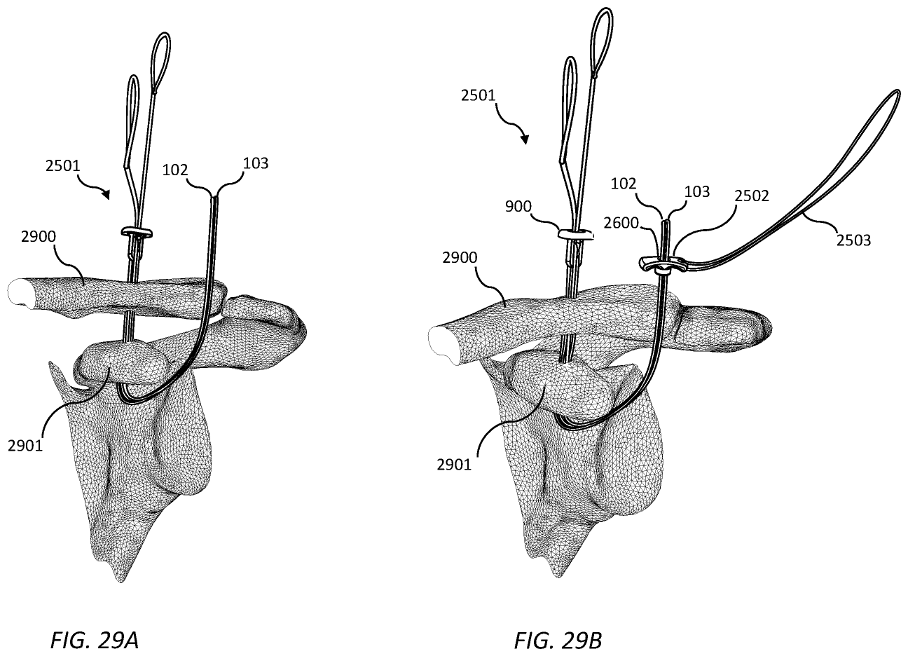

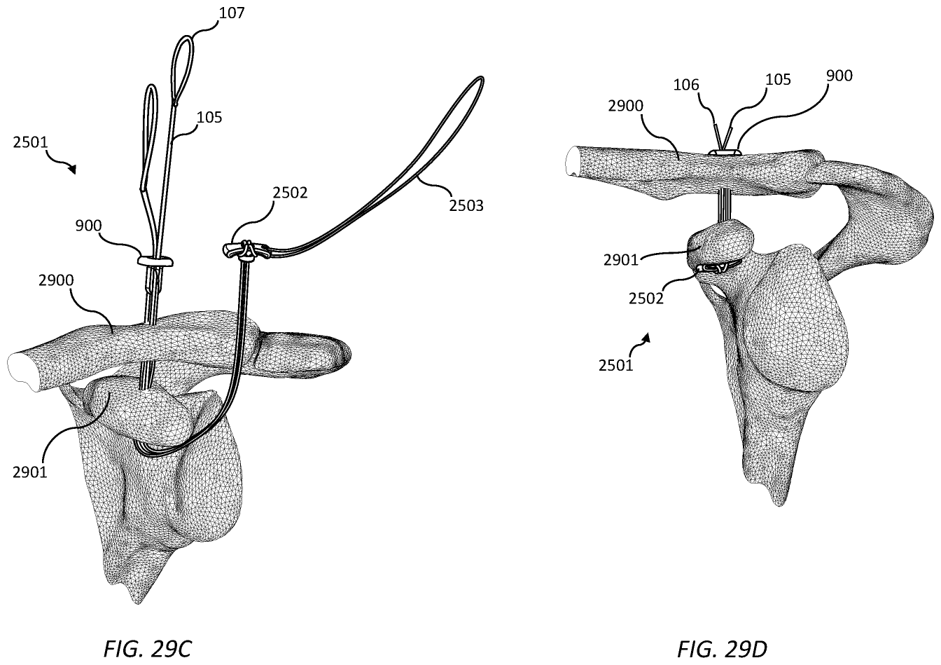

FIGS. 29A-29D illustrate exemplary steps for one use of suspensory fixation system 2500. In this example, FIG. 29A shows adjustable loops 102 and 103 of suspensory fixation device 2501 passed through an aperture formed in a clavicle 2900 from superior to inferior, and then passed through an aperture formed in a coracoid 2901 from superior to inferior with enough length extending from the inferior aspect of coracoid 2901 for the user to access and manipulate adjustable loops 102 and 103. FIG. 29B-29C illustrate rigid member 2502 assembled onto adjustable loops 102 and 103 as described in the exemplary steps of FIG. 27. FIG. 29D shows the final adjusted position of adjustable loops 102 and 103. In this example, haul 105 has a pulling force applied by manipulating grasping loop 107 in a direction away from rigid member 900. As adjustable loops 102 and 103 reduce to a smaller diameter, a compressive force will be generated between rigid member 900 and rigid member 2502, and compress the bony structures therebetween. Haul 105 and transport limb 106 may then be trimmed to remove excess material as illustrated.

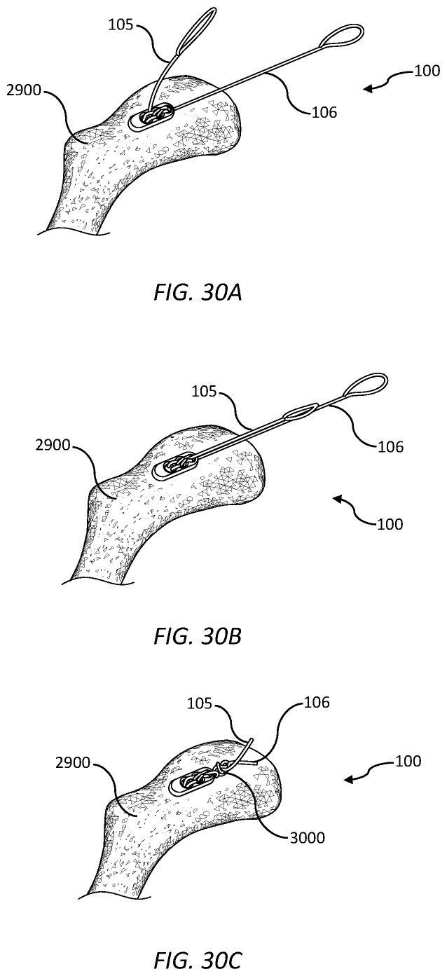

Though not required, it may be a user preference to apply a series of back-up knots to suspensory fixation devices after the length adjustment step. In this example, FIG. 30A shows rigid member 101 of suspensory fixation device 100 approximated against the superior surface of clavicle 2900 and spanning the aperture in the bone with the adjustable loop length adjustment completed. FIG. 30B highlights that when haul 105 is brought in proximity to transport limb 106 for the purposes of knot tying, the two limbs are on opposite sides of rigid member 101. FIG. 30C illustrates the configuration and position of security knot stack 3000 relative to rigid member 101. The user may tie one or more half-hitch or other non-sliding knots to secure haul 105 to transport limb 106. Advantageously, knot stack 3000 extends in a direction generally parallel to the long axis of rigid member 101, reducing the contribution to the overall height of the implant from the bone surface.

What is claimed is:

1. A suspensory fixation device, comprising:
a block at a first end thereof, the block comprising a rigid fixation member having a first end and a second end and a plurality of apertures therebetween forming at least one sheave and at least one becket;
a tackle comprising a single fall reeved through the apertures of the standing block to form a set of at least two adjustable loops at the second end thereof, the fall comprising a flexible cable with a first end and a second end, wherein at least one portion of the fall engages at least one sheave of said standing block;
a sliding knot, formed in the fall and adjustable in one direction, the sliding knot having at least one portion of the fall forming a locking loop on one side of the rigid fixation member and engaging with a becket, both ends of the fall passing through the locking loop, wherein the portion engaging the becket functions as the standing part in a block and tackle system, wherein the portion of the fall forming the locking loop also engages a becket, wherein the locking loop is formed prior to engaging the becket; and
a haul, wherein the haul is operable to adjust the sliding knot;
wherein the adjustment of the sliding knot reduces the diameter of the set of loops,
wherein the portion of the reeved fall engaging a becket on the rigid fixation member forms the standing part of the block and tackle system.

2. The suspensory fixation device of claim 1, wherein any sheave in the rigid fixation member may also function as a becket.

3. The suspensory fixation device of claim 1, the knot further comprising a transport limb, wherein a force applied to the transport limb will not cause the set of loops to change diameter.

4. The suspensory fixation device of claim 1, further comprising:
a second rigid fixation member at the second end thereof,
wherein the fall is reeved through apertures and over at least one sheave of the second fixation member;
wherein the second rigid fixation member forms a running block in the block and tackle system.

5. The suspensory fixation device of claim 1, the rigid fixation member curved along its long axis, wherein the radius of curvature allows the rigid fixation member to be congruent with an anatomic structure when approximated to said anatomic structure.

6. The suspensory fixation device of claim 1, further comprising a needle coupled to at least one adjustable loop of the set.

7. The suspensory fixation device of claim 6, wherein the needle is moveable on at least one adjustable loop.

8. The suspensory fixation device of claim 1, further comprising a grasping element formed on at least one end of the fall.

9. The suspensory fixation device of claim 3, wherein the transport limb extends from a side of the rigid fixation member opposite of the haul.

10. A suspensory fixation device, comprising:
a rigid fixation member;
a flexible cable fashioned into at least one adjustable loop, wherein the rigid fixation member coupled to at least one adjustable loop at the first end; and
a graft support member comprising a flexible cable integrated onto at least one adjustable loop at the second end, the flexible cable having a first end and a second end, and a hollow portion therebetween;
wherein a portion of at least one adjustable loop is contained within said hollow portion of the graft support member.

11. The suspensory fixation device of claim 10, the graft support member further comprising a needle affixed to at least one end of the flexible cable.

12. The suspensory fixation device of claim 10, the graft support member further comprising:
a closed loop formed by joining the ends of the graft support member; and
a needle coupled to said closed loop.

13. The suspensory fixation device of claim 12, wherein the needle is movable on the closed loop.

14. A method of forming an adjustable, self-locking knot, comprising:
reeving a single fall through the apertures of at least one rigid fixation member to form a tackle, the rigid fixation member comprising a first end and a second end and a plurality of apertures therebetween through a first side to a second side forming at least one sheave and at least one becket, the fall comprising a flexible member with a first end and a second end;
forming a locking loop with a portion of the fall on one side of the rigid fixation member through which the ends of the fall are passed;
engaging a portion of the fall with a becket on the rigid fixation member, wherein the engaged portion functions as the standing part in a block and tackle system; and
passing both ends of the fall through the locking loop;
wherein the end of the fall forming the locking loop also engages a becket,
wherein the locking loop is formed prior to the end engaging the becket.

15. A method of coupling a suspensory fixation button to a set of adjustable flexible loops, comprising:
passing a set of adjustable loops through an aperture on a suspensory fixation button from a first side to a second side, the suspensory fixation button comprising a rigid body having a first side and a second side and at least a first lobe and a second lobe, the lobes extending radially from the body and at least one aperture formed through the body between the first lobe and the second lobe from the first side to the second side, the set of adjustable loops comprising a first adjustable loop and a second adjustable loop;
passing the first adjustable loop over an edge of the first lobe to arrive on the first side of the first lobe; and
passing the second adjustable loop over an edge of the second lobe to arrive on the first side of the second lobe.

16. The method of claim 15, further comprising, passing the set of adjustable loops through tissue.

17. The method of claim 15, the suspensory fixation button further comprising a sleeve extending from the first side of the rigid body concentric with the aperture.

18. The method of claim 15, the suspensory fixation button comprising at least one lobe having a radius of curvature; wherein said lobe forms a concave geometry on the first side of the device.

19. The method of claim 15, the suspensory fixation button further comprising a retrieval means, wherein the retrieval means comprises a flexible member connected to the suspensory fixation button.
